(19) United States
(12) Patent Application Publication
(10) Pub. No.: US 2020/0056170 A1
(43) Pub. Date: Feb. 20, 2020

(54) NUCLEIC ACID APTAMER SCREENING METHOD BASED ON THE LOCALIZED SURFACE PLASMON RESONANCE TECHNOLOGY

(71) Applicant: HENAN ACADEMY OF AGRICULTURAL SCIENCE, Henan (CN)

(72) Inventors: Gaiping ZHANG, Henan (CN); Fangyu WANG, Henan (CN); Ruiguang DENG, Henan (CN); Guangxu XING, Henan (CN); Junfang HAO, Henan (CN); Xiaofei HU, Henan (CN); Qiuying YU, Henan (CN); Jing WANG, Henan (CN); Yubao ZHI, Henan (CN); Dong ZHAO, Henan (CN); Xibao SHI, Henan (CN); Man TENG, Henan (CN)

(21) Appl. No.: 16/349,853

(22) PCT Filed: Dec. 12, 2016
(86) PCT No.: PCT/CN2016/109514

§ 371 Date: May 14, 2019

(30) Foreign Application Priority Data
Nov. 15, 2016 (CN) 201611004815.2

(51) Int. Cl. C12N 15/10; G01N 21/552

(52) U.S. Cl. CPC C12N 15/1048; G01N 21/554

(57) ABSTRACT

Provided is a nucleic acid aptamer screening method based on the localized surface plasmon resonance technology, falling within the fields of molecular recognition and nucleic acid aptamer screening. The method screens out a nucleic acid aptamer that can specifically bind to a target mainly with the aid of a localized surface plasmon resonance personal molecular interaction analyzer. The screening method comprises: taking a nano-gold chip as a medium, fixing the target on the medium, and then carrying out the visualized screening of the nucleic acid aptamer by taking the nucleic acid aptamer as a recognition element. With the aid of the LSPR-SELEX technique, the method does not require any marker during the process of detection by using a solid chip, and maintains the spatial structure and biological activity of the nucleic acid aptamer at the maximum. Compared to the traditional nucleic acid aptamer screening method, the LSPR-SELEX sensing technology is simple to operate and has a high sensitivity, and is less time-consuming (15 min) and has a quick response speed. The greatest advantage lies in that the interaction data is represented on-line in real time, and the affinity between molecules of each round can be acquired quickly and accurately.

### TECHNICAL FIELD

The present invention belongs to the field of molecular recognition and nucleic acid aptamer screening, and the present invention screens out a nucleic acid aptamer that can specifically bind to a target mainly with the aid of a Localized Surface Plasmon Resonance technology (LSPR) personal molecular interaction analyzer.

### BACKGROUND ART

Localized Surface Plasmon Resonance technology (LSPR) is a new, highly sensitive method for the analysis of protein and molecular interactions. Since biomolecules are easily deposited on the surface of nanoparticles, ultrafine particles of 1 to 100 nm, nano-gold particles (AuNPs), are usually used as an LSPR sensing layer and fixed on the surface of the metal film. LSPR's nano-gold sensor (1.5 mm²) is smaller than the continuous gold film used in traditional SPR, and can produces a strong resonance absorption peak in the visible range, making the local refractive index around the particle highly sensitive, so thus the LSPR has a shorter response time and higher detection sensitivity. LSPR can be used not only for the unlabeled real-time monitoring of reactions between many types of biomolecules, but also for the accurate, sensitive and rapid detection of various biochemical indicators. In view of the excellent performance of LSPR technology, it can be widely applied to the fields of food, environment and molecular biology and the like, and will directly become the leading technology for real-time observation of interaction between biomolecules.

Nucleic acid aptamer is a single-stranded DNA or RNA sequence having a length of less than 100 nt, and is a aptamer that is capable of sensitively and specifically binding to a target and is screened from random oligonucleotide library in vitro using new combinatorial chemistry technology of Systematic Evolution of Ligands by Exponential Enrichment (SELEX). When a target molecule is present, the aptamer forms a specific target substance binding site by its own special and stable three-dimensional folding, which allows the aptamer to distinguish the structurally similar substances with only subtle differences in structure. Nucleic acid aptamer, also known as “artificial antibody”, has strong affinity, good stability, non-immunogenicity, and is easy to be modified and labeled. Nucleic acid aptamer has been widely used in three fields of detection, separation and purification and medical treatment.

### SUMMARY OF THE INVENTION

With the aid of LSPR sensing technology, the present invention performs two rounds of screening using the designed oligonucleotide library, and finally obtains clones of different sequences. Further study on the affinity and specificity of the nucleic acid aptamer, and the sequence having the highest affinity and strongest specificity are selected as the ideal aptamer for study. With the aid of LSPR-SELEX technique of the present invention, it can be used to perform nucleic acid aptamer screening on targets of different chips.

In order to achieve the above object, the present invention adopts the following technical solutions:

A nucleic acid aptamer screening method based on the localized surface plasmon resonance technology, wherein, taking a nano-gold chip as a medium, fixing the target on the medium, and then carrying out the visualized screening of the nucleic acid aptamer by taking the nucleic acid aptamer as a recognition element.

The target includes, but not limited to, a small molecule, a protein or a virus.

Specifically, firstly, the oligonucleotide library is diluted with PBS buffer solution as a sample, and then injected; secondly, the system is washed with PBS buffer solution to remove the weakly bound or unbound oligonucleotide molecules, then injection system is washed with water and evacuated with air to prevent the sample from adsorbing on the inner wall of the sample tube; finally, the NaOH regeneration buffer solution is injected, and the nucleotide molecule that specifically binds to a target is eluted and recovered for PCR amplification, so as to prepare a single strand, and obtain a single-chain secondary library; the single-chain secondary library is used for the next round of screening, and screening and PCR amplification are repeated until the nucleic acid aptamer of interest is screened.

Prior to injection, the PBS buffer solution is run until the instrument reaches a stable signal baseline.

The flow rate of injection is 20 μL/min, and the time is 5 min.

The flow rate of the PBS buffer solution during washing is 150 μL/min, and the time is 5 min.

The flow rate of the NaOH regeneration buffer solution during elution is 20 μL/min.

The concentration of the NaOH regeneration buffer solution is 10 mM.

The concentration of the PBS buffer solution is 10 mM.

The screened nucleic acid aptamers can be used for quantitative and qualitative detection of the target.

The beneficial effects of the present invention are as follows:

(1) With the aid of the LSPR-SELEX technology, the present invention does not require any marker during the process of detection by using a solid chip, and maintains the spatial structure and biological activity of the nucleic acid aptamer at the maximum. Taking SA as an example, the present invention uses the LSPR-SELEX technique to screen the nucleotide sequence that specifically binds to SA, and obtains a nucleic acid aptamer SBA with a total length of 114 bp; the affinity characterization is carried out by combining CE experiments, and the result proves that the sequence can bind well to the SA target

(2) Compared to the traditional nucleic acid aptamer screening method, the LSPR-SELEX sensing technology established by the present invention is simple to operate and has a high sensitivity, and is less time-consuming (15 min) and has a quick response speed. The greatest advantage lies in that the interaction data is represented on-line in real time, and the affinity between molecules of each round can be acquired quickly and accurately.

(3) With the aid of the good regenerative performance of the LSPR sensor chip, the present invention can be reused for 80 to 100 times, and can realize detection of multiple samples with different concentrations, thereby greatly reducing the screening cost.

### BRIEF DESCRIPTION OF THE DRAWINGS

FIG. 1: results of affinity identification for aptamers and targets screened by LSPR-SELEX. Wherein, the ordinate represents the signal value detected by the sensor; the abscissa represents the time of interaction of the sample in the sensor.

Graph A shows the results of affinity identification for aptamers and targets screened in the first round. In the figure, the concentration of nucleic acid aptamers corresponding to the curves from top to bottom are gradually reduced (6 μM, 3 μM, 1.5 μM, 0.75 μM, and 0.375 μM).

Graph B shows the results of affinity identification for aptamers and targets screened in the second round. In the figure, the concentration of the nucleic acid aptamers corresponding to the curves from top to bottom are gradually decreased (10 μM, 5 μM, 2.5 μM, 1.25 μM, and 0.625 μM).

FIG. 2: shows the spatial structure of SBA.

FIG. 3: CE-SELEX characterization of SBA aptamers.

A: electrophoretic migration of SA (250 μg/mL); B: electrophoretic migration of SBA (150 μg/mL); C: electrophoretic migration of SBA (75 μg/mL)+SA (125 μg/mL); D: electrophoretic migration of BSA (250 μg/mL); and E: electrophoretic migration of BSA (125 μg/mL)+SBA (75 μg/mL).

### SPECIFIC MODES FOR CARRYING OUT THE EMBODIMENTS

Specific embodiments of the present invention will be further described in detail below with reference to the Examples.

### Example 1: Screening Method of LSPR-SELEX

(1) An oligonucleotide library (a fixed sequence containing 20 bp of upstream primers and 20 bp of downstream primers on both sides, and a random sequence of 80 nt in the middle) was centrifuged at high speed for 1 min. The result was further diluted with ddH₂O to a 100 μM stock solution. The oligonucleotide library was heat denatured (95° C. for 10 min) to destroy the polymer formed between the nucleic acid molecules, and subjected to ice bath for 5 min to avoid polymerization of the nucleic acid. The solution used in the test was filtered using a 0.22 μm microporous filter before use.

(2) Experimental instruments: Open-SPR (Nicoya, Ca), using a nano-gold chip coated using streptavidin (SA), and 10 mM of PBS buffer solution was run at a flow rate of 150 μL/min (4 g of NaCl, 1.45 g of Na₂HPO₄.12H₂O, 0.1 g of KCl, 0.1 g of KH₂PO4, dilute with ddH₂O to a volume of 0.5 L, pH 7.4), until a stable signal baseline was reached.

(3) 100 μM oligonucleotide library stock solution was diluted with PBS buffer solution to a final concentration of 10 μM as a sample. Firstly, 250 μL of the oligonucleotide sample was injected at a flow rate of 20 μL/min, and interacted with the sensor for 5 min. Once at the end of the interaction, the PBS buffer solution was immediately introduced at a flow rate of 150 μL/min so as to wash the system at a high-speed for 5 min, and thereby removing the weakly bound or unbound oligonucleotide molecules; then the injection system is washed with water and evacuated with air to prevent the sample from adsorbing on the inner wall of the sample tube; finally, 250 μL of NaOH regeneration buffer solution (weighing 0.4 g of NaOH, and diluting with ddH₂O to a volume of 1 L) was injected, and interacted with the sensor chip for 5 min at a low flow rate of 20 μL/min, and the nucleotide molecule that specifically binds to a target was eluted and recovered.

### Example 2: PCR Amplification and Preparation of Single Strands

(1) The nucleotide molecule recovered by elution was used as a secondary library for PCR enrichment. The primer sequence was: P1: 5′-TTGACTTGCCACTGACTACC-3′ (SEQ ID NO. 1), P2: 5′-GATGACGACCGACTGACTTC-3′ (SEQ ID NO. 2). The reaction system and procedure for optimizing PCR were as follows: 50 μL reaction systems were as follows: 2 μL of 20 μM P1, 2 μL of 20 μM P2, 25 μL of 2×Taq Master Mix, and 21 μL of secondary library; PCR procedure was as follows: initial denaturation at 95° C. for 5 min; denaturation at 94° C. for 30 s, annealing at 61.7° C. for 30 s, and extending at 72° C. for 30 s (30 cycles); finally extending at 72° C. for 2 min, and storing at 4° C. The amplified product was identified by 2% agarose gel electrophoresis, and the purification was carried out after correct identification, and the product was used as a template for preparing a single-chain secondary library.

(2) Optimize the asymmetric PCR for preparing single-stranded secondary library. 50 μL of reaction system were as follows: 4 μL of 20 μM P2, 25 μL of 2×Taq Master Mix, 19 μL of ddH₂O, and 2 μL of template; PCR procedure was as follows: initial denaturation at 95° C. for 5 min; denaturation at 94° C. for 30 s, annealing at 61.7° C. for 30 s, and extending at 72° C. for 30 s (35 cycles); finally extending at 72° C. for 2 min, and storing at 4° C. The amplified product was electrophoresed by 2% agarose gel at 110 V for 30 min, and the PCR product was purified after correct identification, and the result was used for the next round of screening. At the same time, the PCR product was purified and renatured under the same conditions. After each round of screening, the TraceDrawer data processing and analysis software was used to calculate the KD.

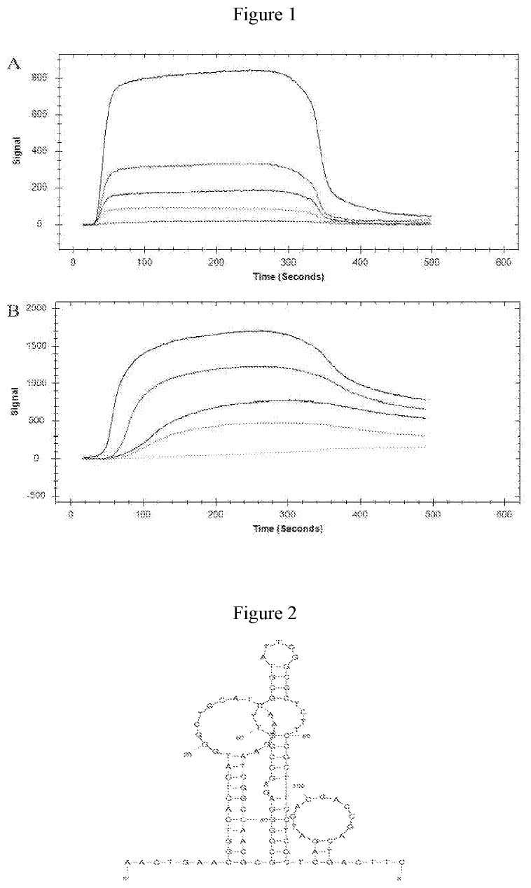

The results showed that: KD value of the nucleic acid aptamers and target screened at the first round of LSPR-SELEX was 107 μM (see FIG. 1A). KD value of the nucleic acid aptamers and target screened at the second round of LSPR-SELEX was 98 μM (see FIG. 1B). This shows that a nucleic acid aptamer with an affinity of 98 μM can be obtained by only two rounds of screening by using LSPR-SELEX technique.

### Example 3: Cloning and Sequencing

The oligonucleotide sequence obtained by the second round of LSPR-SELEX screening was ligated to the T vector for cloning and sequencing, and thereby obtaining aptamer clones of different sequences. Finally, the sequence having the highest affinity and strongest specificity was used as the ideal aptamer for study, and was named as SBA (sequencing results were shown in SEQ ID NO. 3). The results were shown in FIG. 2.

### Example 4: Characterization by Capillary Electrophoresis (CE)

(1) The LSPR-SELEX process was characterized by capillary electrophoresis. Experimental instruments: G7100A (Agilent Technologies, USA); fused silica capillary with an inner diameter of 50 μM, and a length of 56 cm; buffer solution for running and sample diluent were PBS buffer solution (8.5 g of NaCl, 2.2 g of Na₂HPO₄, 0.1 g of NaH₂PO₄, dilute with ddH₂O to a volume of 1 L, pH 7.6)

(2) Before use, 1M NaOH (weighing 4 g NaOH, and diluting with ddH₂O to a volume of 0.1 L), and ddH₂O were used for washing for 5 min and 20 min successively, then the PBS buffer solution was run once to check the baseline. After the baseline was stable, the samples were separated.

(3) Not less than 200 μL per sample, SA, SBA, combination of SA and SBA (incubation at room temperature for 30 min), BSA, combination of BSA and SBA (incubation at room temperature for 30 min) were loaded respectively, the injection pressure was 50 mbar×10 s, separation voltage was 20 KV; and samples were separated for 20 min.

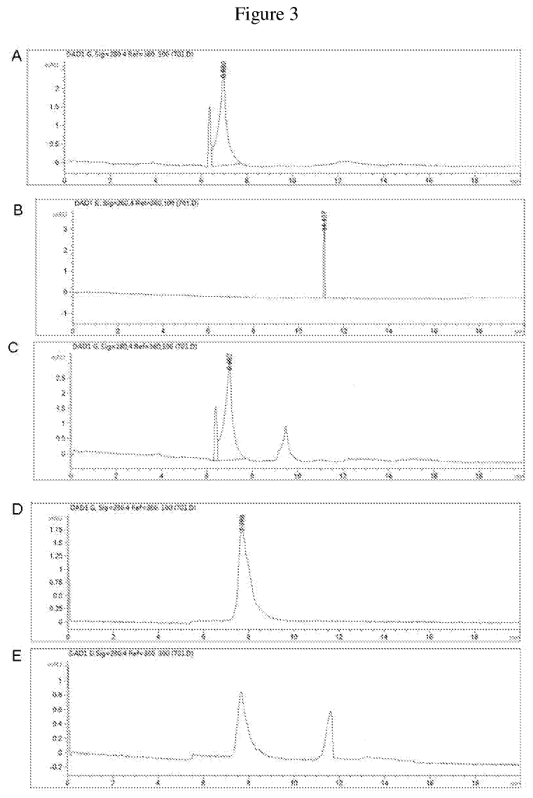

The results of characterization by capillary electrophoresis showed that there was a complex peak after SA+SBA mixed incubation, indicating the interaction between SA and SBA (see FIG. 3A-C). There was no complex peak after BSA+SBA mixed incubation in the control group, indicating that BSA and SBA do not react each other (see FIGS. 3B and D-E).

What is claimed is:

1. A nucleic acid aptamer screening method based on the localized surface plasmon resonance technology, wherein, taking a nano-gold chip as a medium, fixing the target on the medium, and then carrying out the visualized screening of the nucleic acid aptamer by taking the nucleic acid aptamer as a recognition element.

2. The nucleic acid aptamer screening method based on the localized surface plasmon resonance technology according to claim 1, wherein, the target includes, but not limited to, a small molecule, a protein or a virus.

3. The nucleic acid aptamer screening method based on the localized surface plasmon resonance technology according to claim 1, wherein, firstly, the oligonucleotide library is diluted with PBS buffer solution as a sample, and then injected; secondly, the system is washed with PBS buffer solution to remove the weakly bound or unbound oligonucleotide molecules, then injection system is washed with water and evacuated with air to prevent the sample from adsorbing on the inner wall of the sample tube; finally, the NaOH regeneration buffer solution is injected, and the nucleotide molecule that specifically binds to a target is eluted and recovered for PCR amplification, so as to prepare a single strand, and obtain a single-chain secondary library; the single-chain secondary library is used for the next round of screening, screening and PCR amplification are repeated until the nucleic acid aptamer of interest is screened.

4. The nucleic acid aptamer screening method based on the localized surface plasmon resonance technology according to claim 3, wherein, prior to injection, the PBS buffer solution is run until the instrument reaches a stable signal baseline.

5. The nucleic acid aptamer screening method based on the localized surface plasmon resonance technology according to claim 3, wherein, the flow rate of injection is 20 μL/min, and the time is 5 min.

6. The nucleic acid aptamer screening method based on the localized surface plasmon resonance technology according to claim 3, wherein, the flow rate of the PBS buffer solution during washing is 150 μL/min, and the time is 5 min.

7. The nucleic acid aptamer screening method based on the localized surface plasmon resonance technology according to claim 3, wherein, the flow rate of the NaOH regeneration buffer solution during elution is 20 μL/min.

8. The nucleic acid aptamer screening method based on the localized surface plasmon resonance technology according to any one of claims 3 to 7, wherein, the concentration of the NaOH regeneration buffer solution is 10 mM.

9. The nucleic acid aptamer screening method based on the localized surface plasmon resonance technology according to any one of claims 3 to 7, wherein, the concentration of the PBS buffer solution is 10 mM.

10. The nucleic acid aptamer screening method based on the localized surface plasmon resonance technology according to claim 1, wherein, the screened nucleic acid aptamer is used for quantitative and qualitative detection of the target.
